(19) United States
(12) Patent Application Publication
(10) Pub. No.: US 2013/0243906 A1
(43) Pub. Date: Sep. 19, 2013

(54) Method and System of Making a Leavened Dough Composition

(71) Applicant: John C. Phillips, Struthers, OH (US)

(72) Inventor: John C. Phillips, Struthers, OH (US)

(21) Appl. No.: 13/475,951

(22) Filed: May 19, 2012

Related U.S. Application Data
(60) Provisional application No. 61/612,379, filed on Mar. 18, 2012.

(51) Int. Cl. A21D 8/04; C12M 1/00

(52) U.S. Cl. 426/20; 426/19; 435/289.1

(57) ABSTRACT

A method of making a leavened dough composition includes making a sponge with a mixing device, and separating the sponge into a plurality of subsections with a separating apparatus.

This patent claims the benefit of U.S. Provisional Application Ser. No. 61/612,379, filed Mar. 18, 2012, the disclosure of which is incorporated herein by reference in its entirety.

### FIELD

The present disclosure relates to leavened dough compositions and, in particular, to a method and a system for making a leavened dough composition using a sponge and dough process.

### BACKGROUND

Baked goods, such as breads, are made by preparing a dough and then baking the dough in an oven. In a commercial bakery, the dough is typically prepared according to either a two-stage process or a one-stage process. The two-stage process is referred to as the sponge and dough process. The sponge and dough process produces a baked good that some consumers consider to be superior in taste, texture, and aroma, as compared to baked goods prepared using other methods of dough preparation, such as the one-stage process. In particular, the sponge and dough process produces a crumb that is soft and resilient and a crust that is tender, but uniformly thick and strong.

The first stage of making dough with the sponge and dough process is to make a sponge. The sponge (also referred to as a poolish, biga, slurry, chef, levain, or pre-ferment) is made by mixing water, flour, and yeast. Typically, the sponge includes a portion of the total amount of flour and most of the yeast to be used in the dough. After the ingredients of the sponge are mixed, the sponge is allowed to leaven for a predetermined time period, the length of which is dependent on the type of baked good to be made. During leavening, the sponge ferments, a process in which the yeast consumes carbohydrates in the flour and expels various gases including carbon dioxide. The gases expelled by the yeast cause the sponge to increase in volume, a process commonly referred to as “rising.”

The sponge is added to the remaining portion of the flour and the water after the sponge the predetermined time period to form the dough. Depending on the type of baked good to be made, salt, oil, and other ingredients may also be added to the dough.

Next, the dough is typically scaled into subsections, which have a weight suitable for baking. For example, in a commercial bakery the dough is scaled into subsections that have a suitable weight for producing a loaf of bread. Machines deposit the subsections of dough into a pan.

After the subsections of dough have been deposited into a pan, they leaven for another predetermined time period. This second leavening period is referred to as “proofing.” During proofing the subsections of dough are placed in a space of controlled humidity and temperature, which encourages further fermentation. After proofing the twice-leavened dough is ready to be baked.

The sponge and dough process of dough making is not the fastest or most efficient process to produce a dough; accordingly, in some commercial bakeries, the one-stage process of dough making is used. In the one-stage or straight-dough process of dough making the entire portions of water, flour, yeast, and other ingredients, are combined in one step to form the dough directly without forming a sponge. The one-stage process of dough making typically results in a baked good that lacks the complex aroma, flavor, and texture of baked goods made using the sponge and dough process.

What is needed, therefore, is an improvement in the efficiency of using the sponge and dough process in a commercial bakery.

### SUMMARY

According to one embodiment of the disclosure a method of making a leavened dough composition includes making a sponge with a mixing device, and separating the sponge into a plurality of subsections with a separating apparatus.

The method further includes transporting the plurality of subsections with a transporting device. The transporting device includes an endless belt and the method further includes depositing the plurality of subsections onto the endless belt.

The method still further includes (i) depositing the sponge into a trough, (ii) resting the sponge in the trough, and (iii) separating the sponge with the separating apparatus after the resting.

According to the method, the sponge comprises a first portion of flour and a first portion of yeast. The sponge is added to a second portion of flour after the separating to form the leavened dough composition.

According to the method, the leavened dough composition includes a second portion of yeast. The first portion of yeast is at least 98% of a total amount of yeast in the leavened dough composition. The second portion of yeast is less or equal to 2% of the total amount of yeast. The first portion of flour is approximately 70% of a total amount of flour in the leavened dough composition. The second portion of flour is approximately 30% of the total amount of flour in the leavened dough composition

According to the method, the mixing device is a first mixing device, and the method further includes (i) transporting the plurality of subsections to a second mixing device, (ii) adding a portion of flour to the second mixing device, and (iii) mixing the plurality of subsections and the portion of flour in the second mixing device to form the leavened dough composition.

According to another embodiment of the disclosure a method of making a leavened dough composition with a sponge comprised of flour, water, and yeast, includes viding the sponge into a plurality of subsections.

The method further includes (i) depositing at least a portion of the plurality of subsections onto a transport device, which includes an endless belt, (ii) serially transporting the deposited subsections to a mixing device with the transport device, (iii) adding a first portion of flour to the mixing device, (iv) adding a first quantity of water to the mixing device, and (v) mixing the deposited subsections, the first portion of flour, and the first quantity of water in the mixing device to form the leavened dough composition.

The method still further includes resting the sponge before the dividing.

According to the method, the sponge includes only a second quantity of water, a second portion of flour, and a portion of yeast.

According to yet another embodiment of the disclosure a system for making a leavened dough composition includes a mixing device and a separating device. The mixing device is configured to make a sponge. The separating device is configured to separate the sponge into a plurality of subsections.

The system further includes a transporting device configured to transport the plurality of subsections.

According to the system, the mixing device is a first mixing device, and the transporting device transports the plurality of subsections to a second mixing device and/or a third mixing device.

According to the system, the transporting device includes at least one endless belt, and the separating device is further configured to deposit each subsection of the plurality of subsections onto the at least one endless belt.

The system further includes an elevator configured to discharge the sponge into the separating device. The elevator is unsuitable to deposit the sponge into the second mixing device and the third mixing device. In particular, the elevator elevators the sponge to a first predetermined height. The second mixing device includes a sponge opening. The sponge opening is positioned at a second predetermined height. The first predetermined height is less than the second predetermine height.

According to the system, the transporting device extends through at least one of an opening in a wall and an opening in a ceiling.

### BRIEF DESCRIPTION OF THE DRAWINGS

FIG. 1 is a block diagram of a portion of a system for making a leavened dough composition, as described herein;

FIG. 2 is a block diagram of another portion of the system for making a leavened dough composition, as described herein;

FIG. 3 is a flowchart depicting a method of making the leavened dough composition using the system shown in FIGS. 1 and 2; and

FIG. 4 is a block diagram of a portion of a prior art system for making a leavened dough composition.

### DETAILED DESCRIPTION

For the purpose of promoting an understanding of the principles of the disclosure, reference will now be made to the embodiments illustrated in the drawings and described in the following written specification, It is understood that no limitation to the scope of the disclosure is thereby intended. It is further understood that the present disclosure includes any alterations and modifications to the illustrated embodiments and includes further applications of the principles of the disclosure as would normally occur to one skilled in the art to which this disclosure pertains.

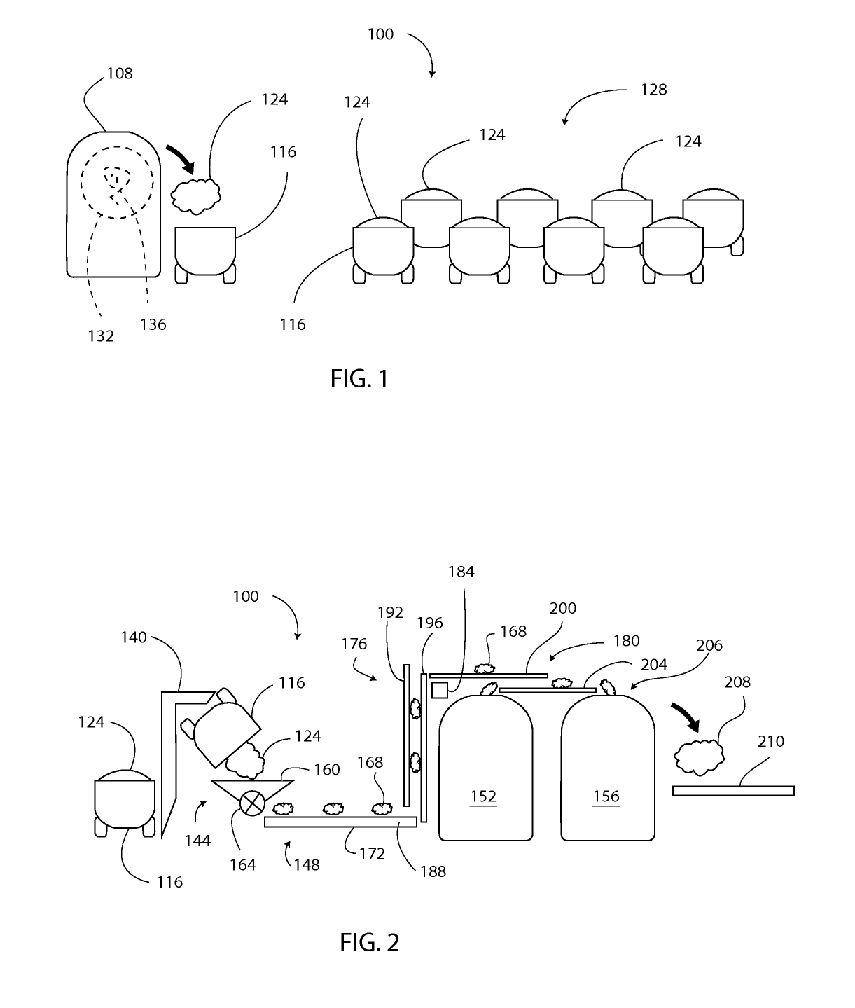

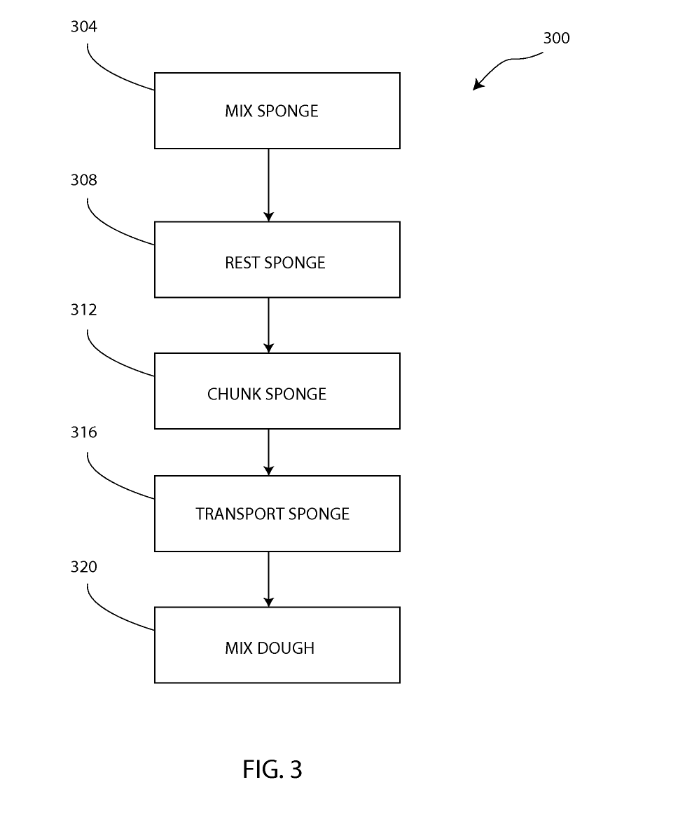

As shown in FIGS. 1 and 2, a system 100 for making a leavened dough composition includes a mixing device 108, numerous troughs 116, and numerous batches of a sponge 124, some of which are positioned in a fermentation area 128. As described herein, the system 100 separates the sponge 124 into easily transportable chunks 168 in order to simplify the dough making process. Each component of the system 100 is descried below followed by a description of a method 300 (FIG. 3) of operating the system.

The mixing device 108, which is also referred to herein as a mixer or as a sponge mixer, receives the ingredients that are used to make the sponge 124. The mixer 108 includes a bowl 132 and an agitator 136, among other components. The bowl 132 holds the ingredients of the sponge 124, which typically weigh between one thousand pounds (1000 lbs.) and fifteen hundred pounds (1500 lbs.). The agitator 136 moves relative to the bowl 132 to sweep and fold the ingredients within the bowl. The bowl 132 is tilt-able in order to discharge the contents (i.e. the sponge 124) from the mixer 108 and into one of the troughs 116 as shown by the bold arrow in FIG, 1. The mixer 108 is additionally or alternatively any mixer as known to those of ordinary skill in the art, including mixers having a greater mixing capacity, mixers having less mixing capacity, and mixers in which the sponge 124 is manually removed therefrom.

The troughs 116 are wheeled containers that hold the sponge 124 and enable the sponge to be moved within the bakery. The troughs 116 are sized to contain the sponge 124 even after it has risen as a result of yeast action. Typically, the troughs 116 are formed from stainless steel, mild steel, or another suitable material. Each of the troughs 116 weighs approximately five hundred pounds (500 lbs.). The troughs 116 are additionally or alternatively any trough as known to those of ordinary skill in the art.

With reference to FIG. 2, the system 100 further includes an elevator 140, a separating device/apparatus 144, a transporting/transport device 148, a mixing device 152, and a mixing device 156. The elevator 140 is a lifting device that lifts and tilts one of the troughs 116 to discharge the sponge 124 contained therein. The elevator 140 is positioned near the separating device 144 so that the discharged sponge 124 falls into the separating device (as shown in FIG. 2). The elevator 140 is additionally or alternatively any elevator known to those of ordinary skill in the art.

The separating device 144, which may also be referred to herein as a chunker, a chunking device, or a separator includes a chute 160 and a cutting device 164. The chute 160 is a funnel-like guide that catches the sponge 124 as it is discharged from the trough 116, The chute 160 guides the sponge 124 to the cutting device 164. The viscosity of the sponge 124 enables it to move to the cutting device 164 by force of gravity alone; accordingly, mechanical assistance is not needed to move the sponge to the cutting device.

The cutting device 164 of the separator 144 cuts, divides, and/or separates the sponge 124 into subsections referred to herein as chunks 168. The chunks 168 weigh approximately forty to fifty pounds (40 to 50 lbs.) and are approximately one foot wide by one foot tall by four feet long (1 ft×1 ft×4 ft). From a typical sponge 124 the cutting device 164 forms approximately twenty (20) to thirty (30) of the chunks 168. The chunks 168 exit the cutting device 164 and are received by the transporting device 148.

The transporting device 148 serially transports the chunks 168 to the mixers 152, 156. The transporting device 148 includes a horizontal section 172, a vertical section 176, a horizontal section 180, and a controller 184. The horizontal section 172 extends between the separator 144 and the vertical section 176. The vertical section 176 extends between the horizontal section 172 and the horizontal section 180. The horizontal section 180 extends between the vertical section 176 and the mixer 152 and the mixer 156. The controller 184 supplies power to the sections 172, 176, 180 according to an electronic program stored within the controller (electric wires and data lines extend from the controller to the sections 172, 176, 180, but are not shown in FIG. 2). The transporting device 148 is described as “serially” transporting the chunks 168 since the chunks are transported in series in single file, similar to bits of data being transmitted serially or current flow through a serial circuit. As a result of the viscosity of the sponge 124, the chunks 168 generally maintain their shape as they are transported by the transporting device 148.

The horizontal section 172 includes an endless belt 188 onto which the chunks 168 are deposited from the cutting device 164 of the separator 144. The controller 184 causes the endless belt 188 to rotate to move the chunks 168 toward the vertical section 176.

The vertical section 176 includes an endless belt 192 and an endless belt 196. The endless belts 192, 196 rotate to transport the chunks 168 from the horizontal section 172 to the horizontal section 180. The chunks 168 are pressed between the endless belts 192, 196 to generate enough friction to lift the chunks. The controller 184 causes the endless belts 192, 196 to move the chunks 168 toward the horizontal section 180.

The horizontal section 180 includes an endless belt 200 and an endless belt 204. The endless belt 200 receives the chunks 168 as they are discharged from the vertical section 176 and deposits the chunks onto the endless belt 204. Accordingly, the controller 184 causes the endless belt 200 to rotate so that the chunks 168 move away from the vertical section 176. The endless belt 204 deposits the chunks 168 into one of the mixer 152 and the mixer 156. In particular, the controller 184 causes the endless belt 204 to rotate in a first direction to deposit the chunks 168 in the mixer 152 and a second direction to deposit the chunks in the mixer 156. The controller 184 may cause chunks 168 to be deposited into the each mixer 152, 156 until its associated bowl (not shown) is full, or the controller may alternately deposit the chunks into the mixers so that one chunk is deposited into the mixer 152 and the next chunk is deposited into the mixer 156, and so on.

Although the transporting device 148 is shown as having a horizontal section 172, a vertical section 176, and a horizontal section 180, the transporting device additionally or alternatively includes any arrangement of sections as may be needed to transport the chunks 168 from the separating device 144 to one or more mixers 152, 156. For example, the transporting device 148 may include an inclined section (not shown), a helical section (not shown), and/or a non-linear section (not shown).

The mixer 152 and the mixer 156 are substantially identical to the mixer 108. The mixers 152, 156 are referred herein as dough mixers since the mixers mix additional ingredients with the sponge 124 to form a dough composition 208.

The system 100 makes a leavened dough composition, referred as dough 208, using the two-stage dough making process. The term “dough” and “dough composition”, as used synonymously herein, refers to a composition including the sponge 124 and one or more of flour, water, and yeast. Accordingly, the sponge 124 is an ingredient of the dough 208. The dough 208 has a consistency that is suitable for handling by dough-handling equipment, as is commonly found in a commercial bakery, i.e. the dough has the proper viscosity, weight, and tackiness to be handled effectively. The dough 208 is baked in an oven to make a leavened baked good, such as bread. To simplify comparison of the dough 208 to the sponge 124, the dough is described as having 100% of the flour, 100% of the water, and 100% of the yeast.

The term “sponge”, as used herein, is a composition that is different than the dough 208. The sponge 124 is a combination of flour, water, and yeast, but as compared to the dough 208 the sponge includes less than 100% of the flour, less than 100% of the water, and at least approximately 98% of the yeast. In an exemplary sponge 124 for making white bread, the sponge contains approximately 70% of the flour as compared to the total amount of flour in the dough (by weight or volume), approximately 65% of the water as compared to the total amount of water in the dough (by weight or volume), and at least 99% of the yeast as compared to the amount of yeast in the dough (by weight or volume). Accordingly, the sponge 124 has a consistency that is different than the dough 208, and may require equipment that is different, or that is setup differently, than the dough-handling equipment. Specifically, the sponge 124 made to the above-described proportions of ingredients is typically much more vicious than the dough 208 that results therefrom and is much tackier than the dough. Additionally, since the sponge 124 is an ingredient of the dough 208, it is not suitable by itself for making a leavened baked good according to the two-stage dough making process.

The system 100 is operable according to the method 300 illustrated by the flowchart of FIG. 3. Beginning with block 304, water, flour, and yeast are added to the mixer 108 in a proportion that is suitable to form the sponge 124. Next, the mixer 108 is activated to mix the ingredients of the sponge 124 into a viscous mass. When the mixer 108 has sufficiently mixed the ingredients, the sponge 124 is discharged into one of the troughs 116.

As shown in block 308, after the sponge 124 is placed in one of the troughs 116, the trough is moved to the fermentation area 128 for predetermined time period during which the sponge rests. The water in the sponge 124 is supplied at a temperature that causes the yeast to activate. During the predetermined time period, the sponge 124 ferments and rises.

With reference to block 312, after the predetermined time period the trough 116 containing the rested sponge 124 is moved to the elevator 140. The elevator 140 discharges the sponge 124 into the separating device 144, which separates the sponge into chunks 168.

Next, as shown in block 316, the transporting device 148 transports the chunks 168 to either the mixer 152 or the mixer 156. Transporting the chunks 168 with the transporting device 148 greatly simplifies and streamlines the process of moving the rested sponge 124 to the dough mixers 152, 156. The elevator 140 is conveniently located with respect to the fermentation area 128 so that the troughs 116 containing rested sponge 124 are moved only a short distance before they are unloaded. Additionally, the transporting device 148 is modular to enable the chunks 168 to be routed anywhere in the bakery, including through an opening in a wall, an opening in the floor, and around obstructions. In particular, the transporting device 148 may be routed through areas in which it is simply not possible or practical to wheel one of the troughs 116.

Thereafter, as referenced in block 320, when a suitable numbers of chunks 168 have been transported to the mixers 152, 156 the remaining ingredients of the dough 208 including one or more of water, flour, and yeast are added to the mixers. The mixers 152, 156 mix the additional ingredients into the chunks of sponge to form the dough 208. Then the mixers 152, 156 discharge the dough 208 onto an endless belt 210.

The endless belt 210 moves the dough 208 to a divider (not shown), which scales the dough into subsections that are specific to the type of baked good to be made. The subsections are deposited into pans and then rest for another predetermined time period in which additional fermentation occurs. After this second resting period, which is referred to as proofing, the dough 208 is baked in an oven to produce the baked good.

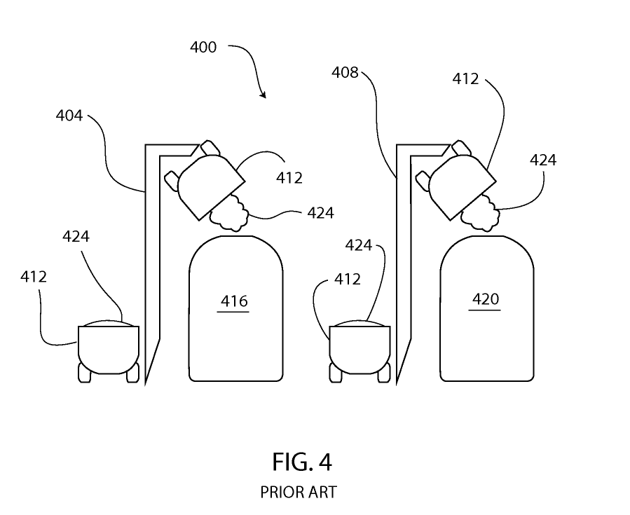

As shown in FIG. 4, a portion of a prior art bakery 400 includes two trough elevators 404, 408 for lifting troughs 412 and two dough mixers 416, 420 for mixing dough (not shown). Additionally, numerous sponges 424 are shown, which are substantially identical to the sponge 124. The elevators 404, 408 lift the troughs 412 so that the sponge 424 can be discharged directly into one of the mixers 416, 420. A separate elevator 404, 408 is required for each of the mixers 416, 420. Generally, each of the elevators 404, 408 is operated by a laborer.

The system 100 offers numerous advantages over the prior art bakery 400. First, the system 100 eliminates one of the elevators 404, 408 and one of the laborers of the bakery 400. Second, the system 100 replaces the two “high-lift” elevators 404, 408 with one “low-lift” elevator 140. The low-lift elevator 140 is capable of lifting the sponge 124 to a predetermined height. The mixers 152, 156 include a sponge opening 206 that is positioned at a second predetermined height. Since the first predetermine height is less than the second predetermined height the elevator 140 is unsuitable to deposit the sponge 124 in the mixers 152, 156. Third, the system 100 prevents a laborer from having to wheel the troughs 116 containing the rested sponge 124 from the fermentation area (128 in FIG. 1) to the dough mixers 416, 420. It is noted that the low-lift elevator 140 is unable to discharge the sponge 124 into the mixers 152, 156, since it is not capable of lifting the sponge to the height of the bowl (not shown) of the mixers 152, 156.

In the prior art bakery 400 the sponge 424 is discharged into the mixers 416, 420 as whole unit, because it was thought that separating the sponge into chunks would adversely affect the resultant baked good and/or adversely affect the production process for at least two reasons. First, it was thought that the sponge 124, 424 would be too viscous to flow through the separator 144 without mechanical assistance. It was determined, however, that the sponge 124, 424 flows through the separator 144 without requiring any mechanical assistance. Second, it was thought that the separator 144 would damage the gluten structure of the sponge 124, 424. In particular, it was thought that the shearing action of separator 144 would reduce the viscoelastic properties of the wheat gluten proteins that are formed when the flour and the water of the sponge 124, 424 are mixed. However, it has been determined that the gluten proteins of the sponge 124, 424 are not noticeably affected by the separating, with the result that the dough 208 is just as strongly structured as dough made from a sponge that is not separated into the chunks 168.

With reference again to the system 100 of FIGS. 1 and 2, separating the sponge 124 into chunks 168 results in numerous benefits to the dough making process. First, the chunks 168 are degassed more effectively by the dough mixer 152, 156 as compared to the degassing characteristics of the sponge 124 as a whole (as is done in the bakery 400). “Degassing” the sponge 124 refers to expelling carbon dioxide from the sponge by sweeping and folding the sponge in the dough mixers 152, 156. A sponge 124 that is more properly “degassed” is more easily mixed with the remaining ingredients of the dough 208. Accordingly, chunking the sponge 124 results in a homogenous dough 208 with a uniform density. Also, less energy is required to degas the sponge 124 after it has been separated resulting in reduced energy costs.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

What is claimed is:

1. A method of making a leavened dough composition, comprising:
making a sponge with a mixing device; and
separating said sponge into a plurality of subsections with a separating apparatus.

2. The method of making a leavened dough composition of claim 1, further comprising:
transporting said plurality of subsections with a transporting device.

3. The method of making a leavened dough composition of claim 2, wherein:
wherein said transporting device includes an endless belt, and
said method further comprises depositing said plurality of subsections onto said endless belt.

4. The method of making a leavened dough composition of claim 3, further comprising:
depositing said sponge into a trough;
resting said sponge in said trough; and
separating said sponge with said separating apparatus after said resting.

5. The method of making a leavened dough composition of claim 1, wherein:
said sponge comprises a first portion of flour and a first portion of yeast, said sponge is added to a second portion of flour after said separating to form said leavened dough composition.

6. The method of making a leavened dough composition of claim 5, wherein:
said leavened dough composition includes a second portion of yeast,
said first portion of yeast is at least 98% of a total amount of yeast in said leavened dough composition,
said second portion of yeast is less than or equal to 2% of the total amount of yeast,
said first portion of flour is approximately 70% of a total amount of flour in said leavened dough composition, and
said second portion of flour is approximately 30% of the total amount of flour in said leavened dough composition.

7. The method of making a leavened dough composition of claim 1, the mixing device being a first mixing device, and the method further comprising:
transporting said plurality of subsections to a second mixing device;
adding a portion of flour to said second mixing device; and
mixing said plurality of subsections and said portion of flour in said second mixing device to form said leavened dough composition.

8. A method of making a leavened dough composition with a sponge comprised of flour, water, and yeast, the method comprising:
dividing said sponge into a plurality of subsections.

9. The method of making the leavened dough composition of claim 8, further comprising:
depositing at least a portion of said plurality of subsections onto a transport device having an endless belt;
serially transporting said deposited subsections to a mixing device with said transport device;
adding a first portion of flour to said mixing device;
adding a first quantity of water to said mixing device; and
mixing said deposited subsections, said first portion of flour, and said first quantity of water in said mixing device to form said leavened dough composition.

10. The method of making the leavened dough composition of claim 9, further comprising:
resting said sponge before said dividing.

11. The method of making the leavened dough composition of claim 10, wherein said sponge includes only a second portion of flour, a second quantity of water, and a portion of yeast.

12. A system for making a leavened dough composition, comprising:
a mixing device configured to make a sponge; and
a separating device configured to separate said sponge into a plurality of subsections.

13. The system for making the leavened dough composition of claim 12, further comprising:
a transporting device configured to transport said plurality of subsections.

14. The system for making the leavened dough composition of claim 13, wherein:
said mixing device is a first mixing device, and
said transporting device transports said plurality of subsections to a second mixing device and/or a third mixing device.

15. The system for making the leavened dough composition of claim 14, wherein:
said transporting device includes at least one endless belt, and
said separating device is further configured to deposit each subsection of said plurality of subsections onto said at least one endless belt.

16. The system for making the leavened dough composition of claim 15, further comprising:
an elevator configured to discharge said sponge into said separating device.

17. The system for making the leavened dough composition of claim 16, wherein said elevator is unsuitable to deposit said sponge into said second mixing device and said third mixing device.

18. The system for making the leavened dough composition of claim 17, wherein:
said elevator elevates said sponge to a first predetermined height,
said second mixing device includes a sponge opening,
said sponge opening is positioned at a second predetermined height, and
said first predetermined height is less than said second predetermined height.

19. The system for making the leavened dough composition of claim 15, wherein said transporting device extends through at least one of an opening in a wall and an opening in a ceiling.
